(19) United States
(12) Patent Application Publication
(10) Pub. No.: US 2017/0045533 A1
(43) Pub. Date: Feb. 16, 2017

(54) METHOD AND MEANS FOR THE NON-INVASIVE DIAGNOSIS OF TYPE II DIABETES MELLITUS

(71) Applicants: UNIVERSITAT LEIPZIG, Leipzig (DE); OHIO UNIVERSITY, Athens, OH (US)

(72) Inventors: Ralf HOFFMANN, GroBposna (DE); Andrej FROLOV, Leipzig (DE); Sandro SPILLER, Leipzig (DE); Yichao LI, Athens, OH (US); Lonnie R. WELCH, Athens, OH (US)

(73) Assignees: UNIVERSITÄT LEIPZIG, Leipzig (DE); OHIO UNIVERSITY, Athens, OH (US)

(21) Appl. No.: 15/101,885

(22) PCT Filed: Dec. 5, 2014
(86) PCT No.: PCT/EP2014/076778

§ 371 Date: Nov. 7, 2016

(30) Foreign Application Priority Data
Dec. 5, 2013 (DE) 10 2013 224 978.0

(51) Int. Cl. G01N 33/68

(52) U.S. Cl. CPC G01N 33/6893; G01N 2800/042; G01N 2440/38

(57) ABSTRACT

The invention relates to a method and means for the non-invasive diagnosis of type II diabetes mellitus. The glycation state is determined in at least one glycation position of selected plasma proteins.

The invention relates to a method and means for the non-invasive diagnosis of type II diabetes mellitus.

Type II diabetes mellitus is a disorder in which, although insulin is present in its target location, the cell membranes, it is not able to function as it should (insulin resistance). In the early years of the disease, the pancreas is able to compensate for this by producing insulin in larger quantities. But eventually, the pancreas is no longer able to maintain the superelevated insulin production rate. Then, the insulin it does produce is no longer sufficient to control the level of sugar in the blood, and diabetes mellitus type II becomes manifest. If insulin resistance is high, the blood sugar level still continues to rise, and in some cases the condition of relative insulin deficiency later progresses to absolute insulin deficiency.

Unlike type I diabetes, type II diabetes is rarely associated with loss of weight, and then only in the case of massively elevated blood sugar levels with more frequent urination and thirst. The early stages are characterised by non-specific symptoms such as fatigue, physical weakness, impaired vision and susceptibility to infections such as frequent bladder inflammation. Since these symptoms are very generalised, it often happens that diagnosis is delayed by years, and is then only made by chance. By this time, the health of the individual concerned may already have been compromised irreparably.

For this reason, diagnosis as early as possible is imperative in order to prevent a manifestation of type II diabetes with suitable treatment methods.

The elevated blood sugar level causes a non-enzymatic reaction of sugars with lipids and proteins, and the formation of Amadori products due to Amadori rearrangement (glycation). Endogenous glycation takes place in the body as well, particularly in the bloodstream. In this process, mainly glucose, fructose and galactose enter into uncontrolled reactions with endogenous proteins with no enzyme participation. The problem in this context is that this effect is cumulative over time, particularly against the background of an elevated blood sugar level, possibly resulting in tissue or cell damage. The glycation of HbA_{1c} is therefore used for long-term monitoring of blood sugar levels in diabetics. HbA_{1c}, also called glycohaemoglobin (GHb), is red blood pigment (haemoglobin), that has been chemically changed by glucose.

A method for measuring a glycated protein in which protease and FAOD act on the glycated protein contained in a sample, and wherein a protease of the aspergillus species is used was disclosed previously in DE 69835268 T2. With this method, a glycated protein can be measured with a high degree of sensitivity and accuracy in a component of a living organism by using a suitable protease which has a usable, enzymatic effect in combination with a FAOD, which can be used appropriately to measure glycated albumin. However, the method can only be used to determine the general glycation of the albumin, not the specific glycation that is necessary for a diagnosis of type II diabetes mellitus.

EP 0 623 216 B1 discloses an antibody which reacts specifically to glycated proteins, wherein human serum albumin is also named as such a protein.

EP 0 230 934 A2 discloses a method in which glycated lysine residues serve as the epitope for antibodies. In this way, it is possible to detect glycated proteins such as human serum albumin, for example.

WO 2013/159025 A1 discloses a method for diagnosing diabetes, wherein the N-glycation pattern of various plasma proteins is examined, and changes in this pattern are used for the diagnosis.

US 2004/0147033 A1 further discloses the use of glycoproteins to diagnosis various diseases.

While the diagnostic methods described above are suitable for detecting glycated proteins, such as human serum albumin, they do not allow a reliable diagnosis of type II diabetes.

The object of the present invention is therefore to describe a method for non-invasive diagnosis of type II diabetes mellitus with which it is possible to establish a reliable, simple diagnosis thereof even in the early stage.

The object is solved with the method according to claim 1. Advantageous variants are described in the dependent claims.

According to the invention, a method for non-invasive diagnosis of diabetes, particularly type II diabetes mellitus is described, wherein the glycation of human plasma proteins is determined in at least one glycation position selected from

Lys 64 in human serum albumin (P02768, SEQ ID No. 31),
Lys 73 in human serum albumin (P02768, SEQ ID No. 31),
Lys 174 in human serum albumin (P02768, SEQ ID No. 31),
Lys 181 in human serum albumin (P02768, SEQ ID No. 31),
Lys 233 in human serum albumin (P02768, SEQ ID No. 31),
Lys 262 in human serum albumin (P02768, SEQ ID No. 31),
Lys 359 in human serum albumin (P02768, SEQ ID No. 31),
Lys 378 in human serum albumin (P02768, SEQ ID No. 31),
Lys 414 in human serum albumin (P02768, SEQ ID No. 31),
Lys 525 in human serum albumin (P02768, SEQ ID No. 31),
Lys 545 in human serum albumin (P02768, SEQ ID No. 31),
Lys 574 in human serum albumin (P02768, SEQ ID No. 31),
Lys 41 in the human Ig kappa chain C region (P01834, SEQ ID No. 32),
Lys 75 in the human Ig kappa chain C region (P01834, SEQ ID No. 32),
Lys 99 in the human Ig kappa chain C region (P01834, SEQ ID No. 32),
Lys 163 in the human fibrinogen beta chain (P02675, SEQ ID No. 33),
Lys 211 in the human fibrinogen beta chain (P02675, SEQ ID No. 33),
Lys 295 in the human fibrinogen beta chain (P02675, SEQ ID No. 33),
Lys 1003 of human alpha-2-macroglobulin (P01023, SEQ ID No. 34),
Lys 1 162 of human alpha-2-macroglobulin (P01023, SEQ ID No. 34),
Lys 683 of human serotransferrin (P02787; SEQ ID No. 35),
Lys 50 in the human Ig lambda chain C region (P01842; SEQ ID No. 36),
Lys 120 of human apolipoprotein A-1 precursor (P02647; SEQ ID No. 37),
Lys 131 of human apolipoprotein A-1 precursor (P02647; SEQ ID No. 37),
Lys 141 of human haptoglobin (P00738; SEQ ID No. 38),
Lys 1325 of human complement C3 precursor (P01024; SEQ ID No. 39),
Lys 71 in the human fibrinogen alpha chain (P02671; SEQ ID No. 40),
Lys 581 in the human fibrinogen alpha chain (P02671; SEQ ID No. 40)
and a determination of the HbA_{1c} level is carried out, wherein the glycation in the glycation positions is subsequently correlated with the HbA_{1c} level.

The P-number after the protein name is the UniProt Identifier (accession number). The sequence positions refer to the sequences that are each stored in the accompanying sequence protocol under the SEQ ID indicated in parentheses. The position in human serum albumin refers to the mature protein, without the signal peptide indicated in the UniProt entry and without any propeptide.

In this context, HbA_{1c} is defined as the stable product created by coupling glucose to the N-terminal valine of the haemoglobin A1 beta chain (International Federation of Clinical Chemistry and Laboratory Medicine). It is still commonly expressed as a percentage (%). The international unit introduced in response to the recommendation of the IFCC is mmol/mol haemoglobin, i.e., a per mille value. This value can also be referred to as HbA_{1c}M to prevent confusion with the percentage value. The conversion formula is as follows:

HbA_{1c} [mmol/mol Hb]=(HbA_{1c}[%]−2.15)×10.929.

HbA_{1c} is determined from full blood by means of an enzyme immunoassay.

The following formulas are used to convert the average blood sugar level to the HbA_{1c} value:

HbA_{1c} [%]=(average blood sugar [mg/dl]+86)/33.3

HbA_{1c} [%]=(average blood sugar(plasma) [mg/dl]+77.3)/35.6

Glycation of human plasma proteins is preferably determined in at least one glycation position selected from
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 33),
Lys 50 in the human Ig lambda chain C region (P01842; SEQ ID No. 36),
Lys 141 of human haptoglobin (P00738; SEQ ID No. 38).

Glycation determination is carried out particularly preferably at least in glycation position Lys 141 of human haptoglobin (P00738; SEQ ID No. 38).

Glycation determination is preferably carried out in two to five, particularly preferably three to five, more preferably all six of the glycation positions listed above, wherein the determination of glycation is made at least in the glycation position Lys 141 of human haptoglobin (P00738; SEQ ID No. 38).

Glycation is preferably determined in at least one sequence selected from SEQ ID nos. 1 to 30. Glycation determination is preferably carried out on two to five, particularly preferably three to five, more preferably all of SEQ ID nos. 1 to 30, wherein glycation determination is carried out at least in SEQ ID no. 27.

The sequences of the glycation positions in human plasma proteins according to the invention are listed in the following table 1:

TABLE 1

| SEQ ID | | | Glycation |
|---|---|---|---|
| No. | Sequence | Protein | position |
| 1 | TC*VADESAENC*DK*SLHTLFGDK | human serum albumin | K64 |
| | | (P02768, SEQ ID No. 31) | |
| 2 | SLHTLFGDK*LC*TVATLR | human serum albumin | K73 |
| | | (P02768, SEQ ID No. 31) | |
| 3 | ETYGEMADC*C*AK*QEPER | human serum albumin | K93 |
| | | (P02768, SEQ ID No. 31) | |
| 4 | ETYGEMoxADC*C*AK*QEPER | human serum albumin | K93 |
| | | (P02768, SEQ ID No. 31) | |
| *5 | AAFTEC*C*QAADK*AAC*LLPK | human serum albumin | K174 |
| | | (P02768, SEQ ID No. 31) | |
| 6 | AAC*LLPK*LDELRDEGK | human serum albumin | K181 |
| | | (P02768, SEQ ID No. 31) | |
| 7 | AEFAEVSK*LVTDLTK | human serum albumin | K233 |
| | | (P02768, SEQ ID No. 31) | |
| 8 | ADLAK*YIC*ENQDSISSK | human serum albumin | K262 |
| | | (P02768, SEQ ID No. 31) | |
| 9 | TYETTLEK*C*C*AAADPHEC*YAK | human serum albumin | K359 |
| | | (P02768, SEQ ID No. 31) | |
| 10 | VFDEFK*PLVEEPQNLIK | human serum albumin | K378 |
| | | (P02768, SEQ ID No. 31) | |
| 11 | K*VPQVSTPTLVEVSR | human serum albumin | K414 |
| | | (P02768, SEQ ID No. 31) | |
| 12 | K*QTALVELVK | human serum albumin | K525 |
| | | (P02768, SEQ ID No. 31) | |
| 13 | EQLK*AVMDDFAAFVEK | human serum albumin | K545 |
| | | (P02768, SEQ ID No. 31) | |
| 14 | K*LVAASQAALGL | human serum albumin | K574 |
| | | (P02768, SEQ ID No. 31) | |
| 15 | VQWK*VDNALQSGNSQESVTEQDSK | human Ig kappa chain C | K41 |
| | | region (P01834, SEQ ID | |
| | | No. 32) | |
| 16 | DSTYSLSSTLTLSK*ADYEK | human Ig kappa chain C | K75 |
| | | region (P01834, SEQ ID | |
| | | No. 32) | |
| 17 | VYAC* EVTHQGLSSPVTK*SFNR | human Ig kappa chain C | K99 |
| | | region (P01834, SEQ ID | |
| | | No. 32) | |
| 18 | QVK*DNENWNEYSSELEK | human fibrinogen beta | K163 |
| | | chain (P02675, SEQ ID | |
| | | No. 33) | |
| 19 | IQK*LESDVSAQMoxEYC*R | human fibrinogen beta | K211 |
| | | chain (P02675, SEQ ID | |
| | | No. 33) | |
| 20 | K*WDPYKQGFGNVATNTDGK | human fibrinogen beta | K295 |
| | | chain (P02675, SEQ ID | |
| | | No. 33) | |
| 21 | SK*AIGYLNTGYQR | human alpha-2- | K1003 |
| | | macroglobulin (P01023, | |
| | | SEQ ID No. 34) | |
| 22 | ALLAYAFALAGNQDK*R | human alpha-2- | K1162 |
| | | macroglobulin (P01023, | |
| | | SEQ ID No. 34) | |
| 23 | K*C*STSSLLEAC*TFR | human serotransferrin | K683 |
| | | (P02787; SEQ ID No. 35) | |
| 24 | ADSSPVK*AGVETTTPSK | human Ig lambda chain- | K50 |
| | | C-region P01842; SEQ ID | |
| | | No. 36) | |
| 25 | AKvVQPYLDDFQK | human apolipoprotein A- | K120 |
| | | 1 precursor (P02647; | |
| | | SEQ ID No. 37) | |
| 26 | K*WQEEMoxELYR | human apolipoprotein A- | K131 |
| | | 1 precursor (P02647; | |
| | | SEQ ID No37) | |
| 27 | AVGDK*LPEC*EAVC*GKPK | human apolipoprotein | K141 |
| | | Haptoglobin (P00738; | |
| | | SEQ ID No. 38) | |
| 28 | SEETK*ENEGFTVTAEGK | human complement C3 | K1325 |
| | | precursor (P01024; SEQ | |
| | | ID No. 39) | |
| 29 | MoxK*GLIDEVNQDFTNR | human fibrinogen alpha | K71 |
| | | chain (P02671; SEQ ID | |
| | | No. 40) | |
| 30 | SSSYSK*QFTSSTSYNR | human fibrinogen alpha | K581 |
| | | chain (P02671; SEQ ID | |
| | | No. 40) | |

The sequence positions refer to the sequences that are each stored in the accompanying sequence protocol under the SEQ ID indicated in parentheses. The positions in human serum albumin refer to the mature protein, without the signal peptide indicated in the UniProt entry and without propeptide.

Glycation takes place at the position of the plasma protein lysine residues (K) indicated in Table 1. Preferably, one Amadori product is formed by glycation on each lysine (K). The sequences may also be alkylated on one or more cysteines (C) following the protocol used for enzymatic cleaving of the plasma or serum sample. Optionally, the sulphur in each of the methionines is oxidised (forming sulphur oxide). In this process, K* stands for fructosamine-modified lysine, C* stands for carbamidomethylated cysteine, and M₀ₓ stands for methionine sulfoxide.

Glycation is determined preferably at two to fifteen, particularly preferably five to ten of the glycation positions listed above, wherein glycation determination is carried out at least on SEQ ID No. 27.

In one embodiment of the invention, specific glycation of the lysine residues
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 33),
Lys 50 in the human Ig lambda chain C Region (P01842; SEQ ID No. 36),
Lys 141 of human haptoglobin (P00738; SEQ ID No. 38).
   takes place with type II diabetes patients in a hyperglycaemic state and in the glycation positions of the sequences according to SEQ ID Nos 5, 11, 14, 18, 24 and 27. Compared with this, in a control group no glycation of any kind was detected in the glycation positions of the above listed lysine residues or sequences with SEQ ID Nos 5, 11, 14, 18, 24 and 27. At the same time, particularly the glycation of lysine residue 141 in SEQ ID No. 27 together with the HbA_{1c} level has high sensitivity of 93.8% and high selectivity of 97.9% for a diagnosis of type II diabetes.

Accordingly, the glycation positions are suitable for use as biomarkers in a diagnosis of diabetes, particularly type II diabetes mellitus and/or for monitoring treatment for diabetes, particularly type II diabetes mellitus. Glycation of these 6 lysines or sequences according to SEQ ID Nos 5, 11, 14, 18, 24 and 27 is an indication of the beginnings of diabetic disease.

In a further embodiment of the invention specific glycation of the lysine residues
Lys 93 (with and without Met(O)) in human serum albumin (P02768, SEQ ID No. 31),
Lys 181 in human serum albumin (P02768, SEQ ID No. 31),
Lys 233 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in human apolipoprotein A-1 (P02647, SEQ ID No. 37),
   takes place with type II diabetes patients in a hyperglycaemic state and in the glycation positions of the sequences according to SEQ ID Nos 3, 6, 7, 10, 12, 23 and 25. Compared with this, in a control group no glycation of any kind was detected in the glycation positions of the above listed lysine residues or sequences with SEQ ID No. 3, 6, 7, 10, 12, 23 and 25.

Accordingly, the glycation positions are suitable for use as biomarkers in early testing for diabetes, particularly type II diabetes mellitus before the manifestation of health and/or for monitoring treatment for diabetes, particularly type II diabetes mellitus. Glycation of these 6 lysines or sequences according to SEQ ID Nos 5, 11, 14, 18, 24 and 27 is an indication of the beginnings of diabetic disease.

In a further embodiment the method comprises the steps of:

Separating the plasma proteins of a blood sample,
Performing enzymatic digestion of the plasma proteins,
Determining the glycation state of at least one of the following lysine residues selected from SEQ ID Nos 1 to 30 and 44 to 46, preferably selected from
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 33),
Lys 50 in the human Ig lambda chain C region (P01842; SEQ ID No. 36),
Lys 141 of human haptoglobin (P00738; SEQ ID No. 38)
   or of a peptide selected from SEQ ID Nos 1 to 30 and 44 to 46, preferably from SEQ-ID Nos 5, 11, 14, 18, 24 and 27, particularly preferably in SEQ ID No. 27
   and determining the HbA_{1c} level, wherein the glycation in the glycation positions is subsequently correlated with the HbA_{1c} level.

In a further embodiment the method comprises the steps of:
Separating the plasma proteins of a blood sample,
preferably: Performing enzymatic digestion of the plasma proteins,
Determining the glycation state of at least one of the following lysine residues selected from SEQ ID Nos 1 to 30 and 44 to 46, preferably selected from
Lys 93 (with and without Met(O)) in human serum albumin (P02768, SEQ ID No. 31),
Lys 181 in human serum albumin (P02768, SEQ ID No. 31),
Lys 233 in human serum albumin (P02768, SEQ ID No. 31),
Lys 378 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in human apolipoprotein A-1 (P02647, SEQ ID No. 37),
or of a peptide selected from SEQ ID No. 1 to 30 and 44 to 46, preferably from SEQ-ID Nos 3, 6, 7, 10, 12, 23 and 25
   and determining the HbA_{1c} level, wherein the glycation in the glycation positions is subsequently correlated with the HbA_{1c} level.

Separation of the plasma proteins is carried out by centrifuging, for example. For the enzymatic digestion step, in general all proteases that ensure breakdown of the plasma proteins are suitable. For example, trypsin can be used for the digestion.

In a variant of the invention, affinity chromatography is carried out after enzymatic digestion to separate glycated peptides and/or for solid phase extraction. For affinity chromatography, a column or preferably magnetic particles is/are used. In one embodiment of the invention, affinity chromatography is conducted in the form of boric acid chromatography. In this case, a specific interaction takes place between the cis-diol groups of the sugar residues in the Amadori peptides and the boric acid. Accordingly, boric acid chromatography is particularly suitable for the effective enrichment of Amadori peptides.

The glycation state is preferably determined by mass spectrometry, FRET (Förster Resonance Energy Transfer), ELBIA (Enzyme Linked Boronate Immunoassay) or immunoassay.

In a further embodiment of the invention, a method for non-invasive diagnosis of diabetes, particularly type II diabetes mellitus, is described, wherein the glycation von human plasma proteins in at least one glycation position selected from
Lys 557 in human serum albumin (P02768, SEQ ID No. 31),
Lys 75 in the human Ig Kappa chain C region (P01834, SEQ ID No. 32),
Lys 131 in the human apolipoprotein A-1 (P02647, SEQ ID No. 37),
Lys 163 in the human fibrinogen beta chain (P02675, SEQ ID No. 33), and
Lys 1 162 of human alpha-2-macroglobulin (P01023, SEQ ID No. 34),
   and the HbA_{1c} level are determined, wherein glycation in the glycation position is subsequently correlated with the HbA_{1c} level.

The P-number after the protein name is the UniProt Identifier. The sequence positions refer to the sequences that are each stored in the accompanying sequence protocol under the SEQ ID indicated in parentheses. The position in human serum albumin refers to the mature protein, without the signal peptide indicated in the UniProt entry and without any propeptide.

Glycation determination is preferably carried out in two to five, particularly preferably three to five, more preferably all five of the glycation positions listed above.

Glycation is preferably determined in at least one sequence selected from SEQ ID nos. 16, 18, 22, 26 or 44. Glycation determination is preferably carried out on two to five, particularly preferably three to five, more preferably all five of the SEQ ID nos. 16, 18, 22, 26 or 44.

The sequences of the glycation positions of human plasm proteins according to the invention are listed in the following Table 2:

| SEQ-ID | | | |
|---|---|---|---|
| No. | Sequence | Protein Annotation | Postion* |
| 44 | AVMDDFAAFVEK*C*C*K | Humanes Serum Albumin | K557 |
| | | (P02768) | |
| 16 | DSTYSLSSTLTLSK*ADYEK | Ig kappa chain C-region | K75 |
| | | (P01834) | |
| 26 | K*WQEEMELYR | Apolipoprotein A-1 | K131 |
| | | (P02647) | |
| 18 | QVK*DNENVVNEYSSELEK | Fibrinogen beta | K163 |
| | | (P02675) | |
| 22 | ALLAYAFALAGNQDK*R | Alpha-2-macroglobulin | K1162 |
| | | (P01023) | |

In this context, glycation takes place the position of the lysine residues (K*) on the plasma proteins listed in Table 1. Preferably, one Amadori product is formed by glycation on each lysine (K). The sequence with SEQ ID No. 44 may also be alkylated on one or more cysteines (C*) following the protocol used for enzymatic cleaving of the plasma or serum sample. Optionally, the sulphur in the methionines Met3 in SEQ ID No. 44 and/or Met6 in SEQ ID No. 26 are each oxidised (forming sulphur oxide).

Surprisingly, it was found that specific glycation of the lysine residues
Lys 557 in human serum albumin (P02768, SEQ ID No. 31),
Lys 75 in the human Ig Kappa chain C region (P01834, SEQ ID No. 32),
Lys 131 in the human apolipoprotein A-1 (P02647, SEQ ID No. 37),
Lys 163 in the human fibrinogen beta chain (P02675, SEQ ID No. 33), and
Lys 1162 of human alpha-2-macroglobulin (P01023, SEQ ID No. 34)
   takes place with type II diabetes patients in a hyperglycaemic state and in the glycation positions of the sequences according to SEQ ID Nos 16, 18, 22, 26 or 44. Compared with this, in a control group no glycation of any kind was detected in the glycation positions of the above listed lysine residues and sequences with SEQ ID Nos 16, 18, 22, 26 or 44. Accordingly, the glycation positions are suitable for use as biomarkers in a diagnosis of diabetes, particularly type II diabetes mellitus and/or for monitoring treatment for diabetes, particularly type II diabetes mellitus. Glycation of these 5 lysines or sequences according to SEQ ID Nos 16, 18, 22, 26 or 44 is an indication of the beginnings of diabetic disease.

In a further embodiment, the method comprises the following steps:

Separating the plasma proteins of a blood sample,
preferably: Performing enzymatic digestion of the plasma proteins,
Determining the glycation state of at least one of the following lysine residues selected from:
Lys 557 in human serum albumin (P02768, SEQ ID No. 31),
Lys 75 in the human Ig Kappa chain C region (P01834, SEQ ID No. 32),
Lys 131 in the human apolipoprotein A-1 (P02647, SEQ ID No. 37),
Lys 163 in the human fibrinogen beta chain (P02675, SEQ ID No. 33), and
Lys 1 162 of human alpha-2-macroglobulin (P01023, SEQ ID No. 34)
or of a peptide selected from a sequence from SEQ ID Nos 16, 18, 22, 26 or 44
and determining the HbA_{1c} level, wherein glycation in the glycation positions is subsequently correlated with the HbA_{1c} level.

Separation of the plasma proteins is carried out by centrifuging, for example. For the enzymatic digestion step, in general all proteases that ensure breakdown of the plasma proteins are suitable. For example, trypsin can be used for the digestion.

In a variant of the invention, affinity chromatography is carried out after enzymatic digestion to separate glycated peptides and/or for solid phase extraction. For affinity chromatography, a column or preferably magnetic particles is/are used. In one embodiment of the invention, affinity chromatography is conducted in the form of boric acid chromatography. In this case, a specific interaction takes place between the cis-diol groups of the sugar residues in the Amadori peptides and the boric acid. Accordingly, boric acid chromatography is particularly suitable for the effective enrichment of Amadori peptides.

The glycation state is preferably determined by mass spectrometry, FRET (Förster Resonance Energy Transfer), ELBIA (Enzyme Linked Boronate Immunoassay) or immunoassay.

The method preferably also comprises determination of the glycation state of at least of the following lysine residues selected from:

| Protein | Position | SEQ ID No. |
|---|---|---|
| Human serum albumin (P02768) | K359 | 31 |
| Human serum albumin (P02768) | K262 | 31 |
| Human serum albumin (P02768) | K64 | 31 |
| Human serum albumin (P02768) | K181 | 31 |
| Human serum albumin (P02768) | K174 | 31 |
| Human serum albumin (P02768) | K51 | 31 |
| Human serum albumin (P02768) | K557 | 31 |
| Human serum albumin (P02768) | K378 | 31 |
| Human serum albumin (P02768) | K233 | 31 |
| Human serum albumin (P02768) | K545 | 31 |
| Human Ig kappa chain C region | K99 | 32 |
| (P01834) | | |
| Human Ig kappa chain C region | K41 | 32 |
| (P01834) | | |
| Human apolipoprotein A-l (P02647) | K120 | 37 |
| Human serotransferrin (P02787) | K683 | 35 |
| Human fibrinogen beta chain | K211 | 33 |
| (P02675) | | |

The sequence positions refer to the sequences that are each stored in the accompanying sequence protocol under the SEQ ID indicated in parentheses. The positions in human serum albumin refer to the mature protein, without the signal peptide indicated in the UniProt entry and without any propeptide.

Glycation is preferably determined at two to fifteen, particularly preferably five to ten of the glycation positions listed above.

For this, determination is preferably carried out of the glycation state of at least one peptide selected from a sequence from the SEQ ID Nos 1 to 30 and 44 to 46 (see also Tables 1 and 2). Glycation takes place at the position of the lysine residues (K*) of the plasma proteins indicated in Table 1 or 2. Preferably, one Amadori product is formed by glycation on each lysine (K). The sequences may also be alkylated on one or more cysteines marked with C* following the protocol used for enzymatic cleaving of the plasma or serum sample. Optionally, the sulphur in each of the methionines is oxidised (forming sulphur oxide). It was found that when in a hyperglycaemic state particularly type II diabetes patients exhibit significantly elevated glycation at the glycation positions in the sequences with SEQ ID Nos 1 to 30 and 44 to 46. Accordingly, the sequences with SEQ ID Nos 1 to 30 and 44 to 46, and preferably at least SEQ ID no. 27 are suitable for supporting a diagnosis based on glycation determination in at least one glycation position selected from SEQ ID Nos 1 to 30 and 44 to 46. The sequences of SEQ ID Nos 1 to 30 and 44 to 46 are listed in Tables 1 and 2. The method also includes the determination of glycation in at least one position selected from SEQ ID Nos 1 to 30 and 44 to 46 and a comparison of glycation both with a control value and with the HbA_{1c} level value, wherein glycation in the glycation positions is subsequently correlated with the HbA_{1c} level.

If the control value is exceeded, the presence of diabetes, particularly type II diabetes mellitus, is confirmed.

Glycation is preferably determined in at least one sequence selected from SEQ ID nos. 1 to 30 and 44 to 46, preferably at least in SEQ ID No. 27. Glycation determination is preferably carried out on two to fifteen, particularly preferably five to ten, SEQ ID nos. 1 to 30 and 44 to 46.

A further object of the invention is also the sequences with SEQ ID nos 1 to 30 and 44 to 46 and the use of a glycated lysine selected from:
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 33),
Lys 50 in the human Ig lambda chain C region (P01842; SEQ ID No. 36),
Lys 141 of human haptoglobin (P00738; SEQ ID No. 38)
Lys 93 (with and without Met(O)) in human serum albumin (P02768, SEQ ID No. 31),
Lys 181 in human serum albumin (P02768, SEQ ID No. 31),
Lys 233 in human serum albumin (P02768, SEQ ID No. 31),
Lys 378 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in the human apolipoprotein A-1 (P02647, SEQ ID No. 37)
   and in the glycation positions of the sequences according to SEQ ID nos 5, 11, 14, 18, 24, 27, 3, 6, 7, 10, 12, 23 and 25 for non-invasive diagnosis von diabetes, particularly type II diabetes mellitus.

A further object of the invention is also the sequences with SEQ ID nos 1 to 30 and 44 to 46 and the use of a glycated lysine selected from:
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 33),
Lys 50 in the human Ig lambda chain C region (P01842; SEQ ID No. 36),
Lys 141 of human haptoglobin (P00738; SEQ ID No. 38)
Lys 93 (with and without Met(O)) in human serum albumin (P02768, SEQ ID No. 31),
Lys 181 in human serum albumin (P02768, SEQ ID No. 31),
Lys 233 in human serum albumin (P02768, SEQ ID No. 31),
Lys 378 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in the human apolipoprotein A-1 (P02647, SEQ ID No. 37),
   and in the glycation positions of the sequences according to SEQ ID nos 5, 11, 14, 18, 24, 27, 3, 6, 7, 10, 12, 23 and 25 for monitoring a treatment for diabetes, particularly type II diabetes mellitus,
   and determining the HbA_{1c} level, wherein the glycation in the glycation positions is subsequently correlated with the HbA_{1c} level.

A further object of the invention is a kit for non-invasive diagnosis of diabetes, particularly type II diabetes mellitus, comprising at least one reagent that has an affinity for at least one antigen that is formed by a peptide, and which comprises at least one of the following lysines selected from SEQ ID nos 1 to 30 and 44 to 46, preferably selected on:
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 33),
Lys 50 in the human Ig Lambda chain C region (P01842; SEQ ID No. 36),
Lys 141 of human haptoglobin (P00738; SEQ ID No. 38)
Lys 93 (with and without Met(O)) in human serum albumin (P02768, SEQ ID No. 31),
Lys 181 in human serum albumin (P02768, SEQ ID No. 31),
Lys 233 in human serum albumin (P02768, SEQ ID No. 31),
Lys 378 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in the human apolipoprotein A-1 (P02647, SEQ ID No. 37),
   and in the glycation positions of the sequences according to SEQ ID nos 5, 11, 14, 18, 24, 27, 3, 6, 7, 10, 12, 23 and 25 and adjacent sequence sections of the corresponding protein.

The antigen preferably has a length of 7 to 25 amino acid residues, and the antigen is particularly preferably formed by a sequence selected from the sequences with SEQ ID nos 1 to 30 and 44 to 46, particularly preferably selected from SEQ ID nos 5, 11, 14, 18, 24, 27, 3, 6, 7, 10, 12, 23 and 25.

The antigen is detected either in the glycated state or in the unglycated state.

The reagent has specific bonding properties with respect to the antigen. In one embodiment of the invention, the reagent is an antibody, an oligonucleotide aptamer or a peptide aptamer. The term antibody includes recombinantly produced antibody fragments such as scFV fragments.

In one embodiment of the invention, the kit further includes at least one immobilised boric acid component for enriching glycated proteins and peptide. Thus, in a first step, glycated proteins and peptides (Amadori proteins or Amadori peptides) may be separated by specific interaction between the boric acid and the cis-diol groups in the sugar residues of the Amadori proteins or Amadori peptides. This is followed by a determination of the Amadori proteins or Amadori peptides (particularly of SEQ ID nos 5, 11, 14, 18, 24, 27, 3, 6, 7, 10, 12, 23 and 25) with the aid of antibodies.

When highly sensitive techniques such as tandem mass spectrometry are used, enrichment is not essential.

In a further embodiment of the invention, the kit includes an ELBIA (Enzyme Linked Boronate Immunoassay). In this case, for example, initially at least one antibody for a sequence selected from SEQ ID nos 1 to 30 and 44 to 46 is immobilised in a reagent vessel (96-well plate; microvessel (1.5 mL)). Then, a sample to be tested is deposited in the microvessel, and a specific interaction takes place between the antibody and the antigen, wherein the antigen has been selected as described previously. The unbonded peptides are removed by washing. Then, a boric acid conjugate, for example boric acid peroxidase conjugate, is added and a peroxidase reaction is initiated by further adding o-phenylene diamine and H2O2. The progress of the reaction may be monitored photometrically at a wavelength of 492 nm.

Another object of the invention is the use of a glycated lysine selected from SEQ ID now 1 to 30 and 44 to 46, preferably selected from:
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 373),
Lys 50 in the human Ig lambda chain C region (P01842; SEQ ID No. 36),
Lys 141 of human haptoglobin (P00738; SEQ ID No. 38)
Lys 93 (with and without Met(O)) in human serum albumin (P02768, SEQ ID No. 31),
Lys 181 in human serum albumin (P02768, SEQ ID No. 31),
Lys 233 in human serum albumin (P02768, SEQ ID No. 31),
Lys 378 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in the human apolipoprotein A-1 (P02647, SEQ ID No. 37),
   and in the glycation positions of the sequences according to SEQ ID nos 5, 11, 14, 18, 24, 27, 3, 6, 7, 10, 12, 23 and 25, preferably SEQ ID No. 27 as biomarkers for the diagnosis of diabetes, particularly type II diabetes mellitus and/or monitoring a treatment for diabetes, particularly type II diabetes mellitus.

A further object of the invention is a biomarker comprising at least one glycated lysine selected from SEQ ID nos 1 to 30 and 44 to 46, preferably selected from:
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 373),
Lys 50 in the human Ig lambda chain C region (P01842; SEQ ID No. 36),
Lys 141 of human haptoglobin (P00738; SEQ ID No. 38)
Lys 93 (with and without Met(O)) in human serum albumin (P02768, SEQ ID No. 31),
Lys 181 in human serum albumin (P02768, SEQ ID No. 31),
Lys 233 in human serum albumin (P02768, SEQ ID No. 31),
Lys 378 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in the human apolipoprotein A-1 (P02647, SEQ ID No. 37),
   and in the glycation positions of the sequences according to SEQ ID nos 5, 11, 14, 18, 24, 27, 3, 6, 7, 10, 12, 23 and 25, preferably SEQ ID No. 27 for the diagnosis of diabetes, particularly type II diabetes mellitus and/or for monitoring a treatment for diabetes, particularly type II diabetes mellitus.

A further object of the invention is a method for diagnosing diabetes, particularly type II diabetes mellitus, comprising the steps of:

Separating the plasma proteins of a blood sample,
Performing enzymatic digestion of the plasma proteins,
Determining the glycation state of at least one of the following lysine residues selected from SEQ ID Nos 1 to 30 and 44 to 46, preferably selected from
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 373),
Lys 50 in the human Ig lambda chain C region (P01842; SEQ ID No. 36),
Lys 141 of human haptoglobin (P00738; SEQ ID No. 38)
Lys 93 (with and without Met(O)) in human serum albumin (P02768, SEQ ID No. 31),
Lys 181 in human serum albumin (P02768, SEQ ID No. 31),
Lys 233 in human serum albumin (P02768, SEQ ID No. 31),
Lys 378 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in the human apolipoprotein A-1 (P02647, SEQ ID No. 37),
   and in the glycation positions of the sequences according to SEQ ID nos 5, 11, 14, 18, 24, 27, 3, 6, 7, 10, 12, 23 and 25, preferably SEQ ID No. 27.

Surprisingly, it was found that a high degree of selectivity can be achieved in a correlation between the determination of the glycation value in a glycation position selected from SEQ ID nos 1 to 30 and 44 to 46 and the HbA_{1c} level. At the same time a structure or pattern may be detected in a dataset of test subjects by Principal Component Analysis, wherein it was revealed after prior classification of the individual patients that a certain percentage of diabetic patients exhibits different behaviour from the other test subjects according to certain parameters.

Principal Component Analysis (PCA) is a technique from multivariate statistics. It is used to structure, simplify and organise large datasets by approximating a large number of statistical variables with a smaller number of linear combinations (the “principal components”) containing as much significant information as possible. Mathematically, a principle axis transformation is performed: the correlation of multidimensional features is minimised by transferring them to a vector space with a new base. The principle matrix which is formed from the eigenvectors of the covariance matrix. Principal Component Analysis is thus problem-specific, because a dedicated transformation matrix must be calculated for each dataset. At the same time, coordinate systems is rotated in such manner that the covariance matrix is diagonalised, i.e., the data is correlated (the correlations are the non-diagonal entries in the covariance matrix). For normally distributed datasets, this means that the individual components of each dataset are statistically unrelated to each other according to the PCA, because the normal distribution is fully characterised by the zeroth (normalising), first (mean) and second moments (covariances).

Principal Component Analysis (PCA) is also used frequently in cluster analysis and to reduce the dimension of the parameter space, particularly when the structure of the data (model) is still unclear. In such circumstances, it is helpful to take advantage of the fact that the PCA rotates the (orthogonal) coordinate system so that the covariance matrix is diagonalised. Moreover, PCA the reorders the coordinate axes (the principal components) in such a way that the first principal component contains the largest share of the total variance in the dataset, the second principal component contains the second largest share, and so on. As was illustrated by the examples in the previous section, the principal components at the back (that is to say the one that only contain a small share of the total variance) can be disregarded entirely without any significant loss of information.

The basic assumption for using PCA for cluster analyse and dimension reduction is as follows: the directions with the largest variance contain the most information.

Subsequently, a cluster analysis was carried out in which it was assumed that diabetics can be divided three subgroups in respect of their glycation patterns. At the same time, it was possible to show that the reduction of the dataset to three dimensions (principal components) allowed the organisation of diabetics into three clusters, which is consistent with clinical observations.

A cluster analysis (clustering algorithm) is understood to be a method for discovering similarity structures in (large) data stocks. The groups of “similar” objects revealed in this way are called clusters, their assignment to groups is called clustering. The similarity groups thus created may be graphic-theoretical, hierarchical, partitioning or optimising in nature.

The properties of the objects to be analysed are treated mathematically as random variables. Usually, they are represented as points in a vector space in the form of vectors, the dimensions of which form the characteristics of the object. Areas in which points occur more frequently (point cloud) are called clusters. In scatter diagrams, the distances between the points or the variance within a cluster function as “proximity dimensions”, which express the similarity—or dissimilarity—among the objects.

A cluster may also be defined as a group of objects that have a minimum offset relative to a calculated focal point. For this, a distance dimension must also be selected. In certain cases, the distances (or conversely the similarities) of the objects from each other is immediately evident, so that they do not have be calculated from their presentation in the vector space.

In order to conduct the cluster analysis for 50 type II diabetes patients, for these purposes the expectation maximization algorithm (EM algorithm for short) was used. Missing values were replaced with mean values of the corresponding feature vector. In this context, the feature space contains such features as Age, Weight, Height, Protein abundance, profile, etc. The method incorporates the assumption that the data is subject to multivariant normal distribution. A cluster number of three was assumed based on prior experience. The results show a very good distribution of the clusters. In addition, a cluster stability test according to the “elbow criterion” was conducted, and it was shown that the maximum number of clusters it could receive is three.

The fundamental idea of the EM algorithm is to start with a randomly selected model, and to alternate the allocation of data to the individual parts of the model (expectation step) improve the parameters of the model for the most recent allocation (maximisation step). In both steps, the quality of the result is improved: in the E-step, the allocation of the points is improved, in the M-step the model is changed so that is matches the data more closely. When no further significant improvement is made, the method is ended.

EM clustering is a cluster analysis method that represents the data in with a “Mixture of Gaussian” model, that is to say as a superposition of normal distributions. This model is initialised randomly or heuristically and is then refined with the general EM principle.

In order to classify 48 type II diabetes patients and 48 control subjects, the decision tree algorithm was used. The feature space was created by the abundance profile of 27 glycated peptide sequences selected from SEQ ID nos 1 to 30 and 44 to 46 and the HbA_{1c} value. The decision tree is an incremental greedy algorithm. Greedy algorithms represent a special class of algorithms and are characterised by the fact that at each stage they choose the following state which promises the greatest gain or the best result at the time the choice is made (e.g., gradient method). In order to make a selection from the following states, an evaluation function is often used. In the present case, a feature was added in each step if an increase in accuracy was achieved. Thus for example specificity of 97.9% was calculated for a combination of HbA_{1c} and Lys-141 in haptoglobin (SEQ ID No. 27). At the same time, when candidates HbA_{1c} (94% specificity & 77% sensitivity) and Lys-141 in haptoglobin (SEQ ID No. 27) (HP K141, 83% specificity & 60% sensitivity) were chosen, it was possible to achieve significantly better sensitivity of 93.8%, and also greater specificity of 97.9% in the diagnosis of type II diabetes by combining the two markers.

It is also beneficial to combine the embodiments described in the preceding text in order to create an optimal version of the invention.

In the following text, the invention will be explained in greater detail with reference to several exemplary embodiments and figures, without limitation thereto.

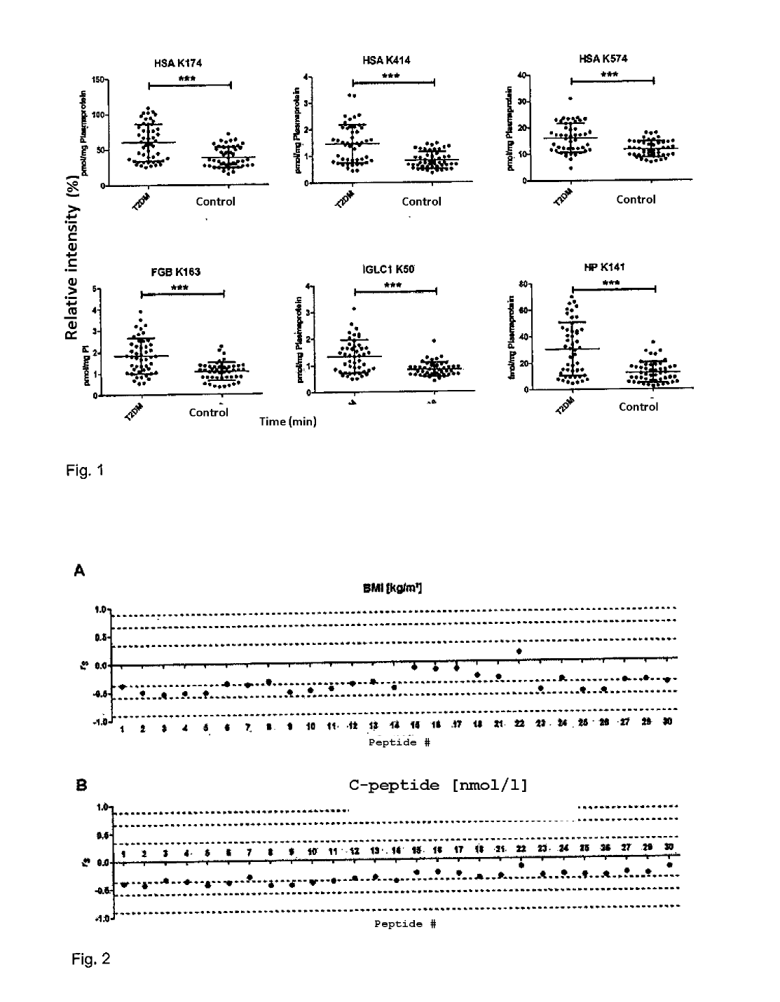

FIG. 1 is a schematic representation of quantification of selected glycated peptides,

FIG. 2 shows a correlation analysis for the correlation of Amadori peptide contents with the values and BMI and C-peptide,

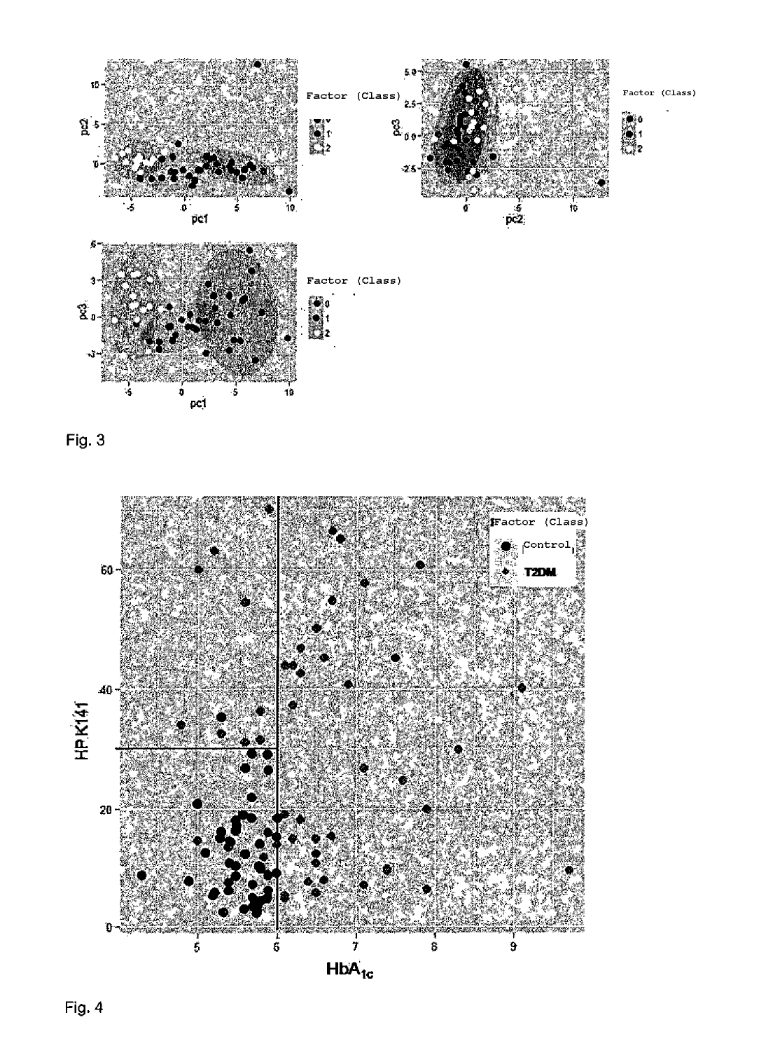

FIG. 3 is a schematic representation of a principal component analysis with division into three subgroups,

FIG. 4 is a schematic representation of HbA_{1c} values plotted against the degree of glycation of HbA_{1c} and Lys-141 in haptoglobin (SEQ ID No. 27),

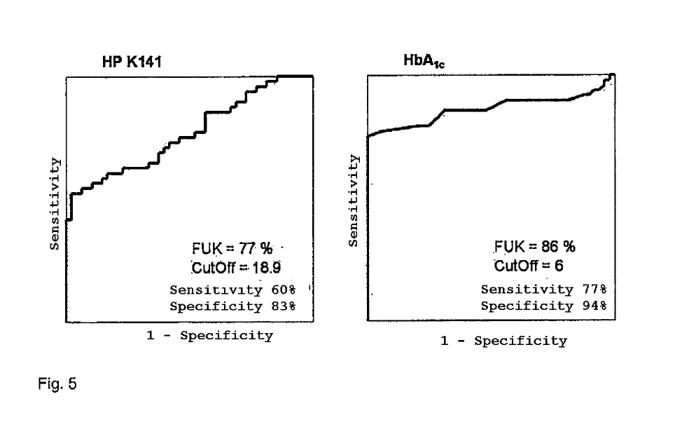

FIG. 5 is a schematic representation of the ROC curves for the degree of glycation of Lys-141 in haptoglobin (HP K141) and HbA_{1c} and

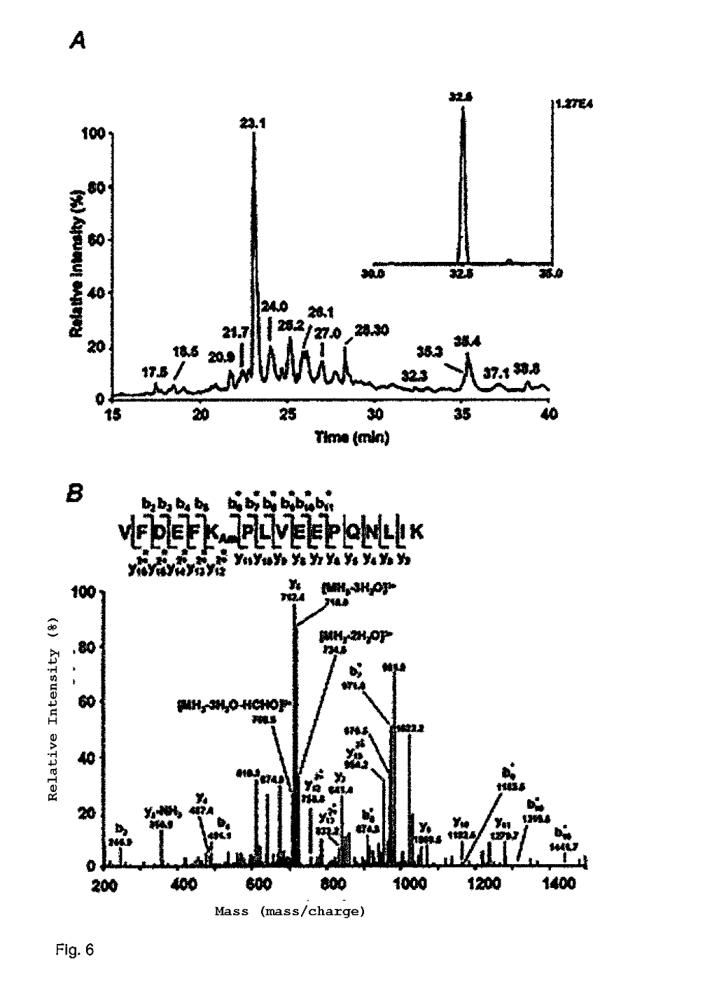

FIG. 6 is a schematic representation of the exemplary determination of glycated peptides by tandem mass spectrometry.

In a first exemplary embodiment, FIG. 1 shows the quantification of selected glycated peptides in tryptic digestion of plasma samples taken from type II diabetics and test control subjects. The differences between the test control subjects and the type II diabetics were significant (p<0.0001).

In a further exemplary embodiment, FIG. 2 shows a correlation analysis according to Spearman. In this, Spearman's correlation coefficient (rₛ) (numeric values displayed as points) results from the correlation of Amadori peptide contents and the values for BMI (A) and C peptide (B) of the individual test subjects.

The rank correlation coefficient according to Spearman is a measure of the strength of a monotonic relationship between two at least ordinally scaled variables. Unlike the correlation coefficient according to Pearson a linear relationship is not a prerequisite for calculating the correlation coefficient according to Spearman. The prerequisites are that the variables to be correlated are scaled at least ordinally, that independent observation pairs are available and that that the relationship to be analysed is monotonic.

For the calculation, first the values of both features are sorted separately in ascending values of the variable and corresponding rank numbers are assigned to each. Then, the difference di is calculated for each value pair (xi,yi). Spearman's correlation coefficient rs is then obtained as follows:

$${r}_{s} = 1 - \frac{6 \cdot {Σ}_{i = 1}^{n} {d}_{i}^{2}}{n \cdot ( {n}^{2} - 1 )}$$

In the above equation, n is the number of observation pairs. If the correlation coefficient rₛ>0, this indicates that a positive relationship exists, if rₛ<0, a negative relationship exists. If rₛ=0, no K relationship exists. The correlation coefficient rₛ may have values between −1 and +1. The closer rₛ is to 0, the weaker the relationship is, the closer rₛ is to −1 or +1 the stronger the relationship.

In a further exemplary embodiment, FIG. 3 shows a cluster analysis based on the assumption that there are three types of diabetes. The resulting clusters are represented as 0, 1 and 2, and each is characterised by internal similarities.

In a further exemplary embodiment, in FIG. 4 HbA_{1c}, values from diabetics and test control subjects are plotted against the degree of glycation of HbA_{1c}, and Lys-141 in haptoglobin. At the same time, a check was also made to determine whether combining different diagnostic parameters with the degree of glycation at the individual glycation points results in increased sensitivity and selectivity for the diagnosis of diabetes. For example, when HbA_{1c}, and Lys-141 in haptoglobin (SEQ ID No. 27) (HP K141) were combined, significantly better sensitivity of 93.8% and even greater specificity of 97.9% were achieved for the diagnosis of type II diabetes. In comparison, FIG. 5 shows the ROC curves for the degree of glycation of Lys-141 in haptoglobin (SEQ ID No. 27) (HP K141) with 83% specificity and 60% sensitivity, and 94% specificity and 77% sensitivity for HbA_{1c}. The Receiver Operating Characteristic (ROC) curve is a method for evaluating and optimising analysis strategies. The ROC curve provides a visual representation of the dependence of efficiency with the error rate for various parameter values. It is an application of signal detection theory.

In a further exemplary embodiment, the determination of glycation in the glycation positions of sequences with SEQ ID nos 1 to 30 is described. Blood samples are drawn from patients with type II diabetes mellitus and non-diabetic subjects and are first centrifuged (9168 g, 30 min, 4° C., Allegra centrifuge 21 R, Beckman Coulter, Krefeld). The supernatant is then diluted by a factor of ten with ammonium bicarbonate (0.1 mol/L, pH 8.0) and then demineralised using a Vivaspin filter (Sartorius Stedim Biotech). The protein concentration obtained was determined by Bradford protein assay. Aliquots with a protein content of 25 μg were reacted with SDS (10% in water, w:v, 2 μL) and TCEP (tris(2-carboxyethyl) phosphine hydrochloride, 50 mmol/L) and diluted with aqueous ammonium bicarbonate solution (50 mmol/L) to a final volume of 20 μL and incubated for 15 min at 60° C. The samples were cooled to room temperature and alkylated in complete darkness (15 min at room temperature) with iodoacetamide (0.1 mol/L, 2.2 μL). Following this, enzymatic digestion with trypsin (25 mg/L in 50 mmol/L ammonium bicarbonate, 50 μL) was carried out at 37° C. overnight. Complete digestion was confirmed by testing the human serum albumin strip using SDS-PAGE.

Aliquots of the digested samples (20 μg) were diluted with cold buffer solution (4° C., 50 mmol/L magnesium acetate; 250 mmol/L ammonium acetate, pH 8.1) to a volume of 300 μL. The samples were placed on a polypropylene column (1 mL; Qiagen) filled with mAPBA (m-aminophenyl boric acid agarose 1 mL, column bed volume).

The Amadori peptides were eluted in two steps with acetic acid (0.1 mol/L, 7 mL and 0.2 mol/L, 1 mL) at 37° C. The eluates were lyophilised.

The lyophilisates were absorbed in a mixture of aqueous acetonitrile solution (20%, v/v, 12.5 μL) and formic acid (0.1%, v/v, 87.5 μL) and introduced into a C18 gel pipette tip (Thermo Fisher Scientific) that had been equilibrated with a aqueous acetonitrile solution (2.5%) containing formic acid (0.1%) (eluent A) for the purpose of solid phase extraction. The gel pipette tip was washed with 150 μL eluent A before the peptides were eluted with an aqueous acetonitrile solution (60%) containing formic acid (0.1%) (eluent B).

The glycation in the glycation positions of the sequences with SEQ-ID nos 1 to 30 was determined using mass spectrometry. For this, the samples were dissolved in an aqueous acetonitrile solution (3%, v/v; 50 μL). Aliquots (10 μL) were placed on a nanoAcquity UPLC Symmetry Trap column (Waters GmbH) (5 μL/min, 5 min) and separated on a nanoAcquity UPLC BEH130 column (Waters GmbH) (30° C.) using a nanoAcquity UPLC system (Waters GmbH). The analytes were eluted to 50% with a linear gradient of 3% (45 min) and from 50% to 80% eluent B (2 min) with a flow rate of 0.4 μL/min. The eluents were analysed by tandem mass spectrometry (LTQ Orbitrap XL ETD; Thermo Fisher Scientific). For the allocation of the peptides, ¹³C-enriched peptides were added to the samples, and the native glycated peptides were quantified in contrast to the added peptides. Alternatively, the peptides were allocated by comparison with the SwissProt database, wherein Amadori peptides were allocated manually. An example of such an allocation is represented in FIG. 1. FIG. 5A also shows a chromatogram of the UPLC with the retention times for the individual analytes, whereas FIG. 5B includes a representation calculated by tandem mass spectrometry of the mass fragments of the individual compounds and their assignment to a sequence.

In a further exemplary embodiment, the determination of glycation in the glycation positions of the sequences of SEQ ID nos 1 to 30 is described. In this process, the concentration of protein in plasma samples taken from type II diabetes mellitus patients and non-diabetic subjects is determined by Bradford assay. Aliquots corresponding to a protein quantity of 1.2 mg were diluted with 1.5 mL ammonium bicarbonate buffer (0.1 mol/L, pH 8.0) and then demineralised (Vivaspin 2 PES MWCO 5 kDa, Sartorius Stedim Biotech). The residue was diluted with ammonium bicarbonate buffer (0.1 mol/L, pH 8.0) to 500 μL. 166.7 μL (400 μg) of the solution was reacted with SDS (0.5% in water, w:v, 20.8 μL) and TCEP (tris(2-carboxyethyl)phosphine hydrochloride, 50 mmol/L, 20.8 μL) and incubated for 15 min at 60° C. The samples were cooled to room temperature and alkylated with iodoacetamide (0.1 mol/L, 22.9 μL) in complete darkness (15 min at room temperature). This was followed by enzymatic digestion with trypsin (25 mg/L in 50 mmol/L ammonium bicarbonate buffer) at 37° C. Initially, 800 μL are added, and after 5 h a further 320 μL enzyme solution is added, and incubation continues for another 12 h. After the digestion, 32 μL of the internal standard solution (concentration optimised mixture) is added and the solution is lyophilised.

The lyophilisates are absorbed in cold buffer (4° C., 50 mmol/L magnesium acetate; 250 mmol/L ammonium acetate, pH 8.1, 20% (v/v) CH₃CN, 100 μL) and diluted to a volume of 500 μL with loading buffer (4° C., 50 mmol/L magnesium acetate; 250 mmol/L ammonium acetate, pH 8.1). The samples are placed on a polypropylene column (1 mL; Qiagen) filled with mAPBA (m-aminophenyl boric acid agarose 1 mL, column bed volume). After a washing step (15 mL loading buffer) the Amadori peptides were eluted at 37° C. in two steps with acetic acid (0.1 mol/L, 8 mL and 0.2 mol/L, 2 mL) and freeze dried.

The lyophilisates are absorbed with an aqueous solution of acetonitrile solution (0.1%, (v/v), formic acid (60%, (v/v), 200 μL) and the concentration of the acetonitrile in solution is reduced successively with 0.1% (v/v) formic acid to 5% (v/v). An Oasis HLB cartridge (30 mg, 1 cc, Waters) was equilibrated with methanol (1 mL) and 0.1% (v/v) aqueous formic acid (1 mL) before the sample was applied and the peptides were eluted with aqueous acetonitrile (both 333 μL) in mixture proportions of 40% (v/v), 60% (v/v) and 80% (v/v) with an addition of formic acid (0.1% (v/v)). The combined eluates are freeze dried.

The glycation positions of the sequences with SEQ-ID nos 1 to 30 are determined by mass spectrometry. For this, the lyophilisates are absorbed with an aqueous solution of acetonitrile (0.1% (v/v) formic acid, 60% (v/v), 10 μL) and the concentration of the acetonitrile content in solution is reduced successively with 0.1% (v/v) aqueous formic acid to 5% (v/v) (final volume of the solution: 120 μL). Aliquots (84 μL) of the solutions were applied to a C18 column (AdvanceBio Peptide Map, 150 mm×2.1 mm, 2.7 μm particle size; Agilent Technologies, Waldbronn, Germany) and separated using a Waters 2695 Alliance Separation Module (Waters GmbH, Eschborn, Germany) (60° C.). Eluent A was 0.1% (v/v) aqueous formic acid and eluent B was 0.1% (v/v) formic acid in CH3CN. The column was equilibrated (3% eluent B) and the peptides were eluted with a multistage linear gradient 3 min after the injection: 3 to 10% eluent B in 1 min, 10 to 20% eluent B in 10 min, 20 to 95% eluent B in 8 min. The flow rate was 300 μL/min. The eluate was analysed on-line on a QqLIT mass spectrometer (4000 QTRAP LC-MS/MS System, AB Sciex, Darmstadt, Germany) with a Turbo V ion source in positive ion mode. Data collection was based on multiple reaction monitoring (MRM) with three specific Q1/Q3-m/z regions for each analyte. The analytes were quantified on the basis of peak integration in the extracted ion chromatogram (extracted ion chromatogram, XIC) and internal standardisation.

An overview of the glycation positions that were calculated is shown in Table 1. Besides the 5 specific glycation positions in the sequences with SEQ ID nos 5, 11, 14, 18, 24, 27, 3, 6, 7, 10, 12, 23 and 25, other glycation positions with significantly elevated glycation were detected in glycation positions SEQ ID nos 1 to 30 and 44 to 46 for type II diabetes mellitus. These sequences might be used to support a diagnosis based on glycation detected in at least one glycation position selected from the sequences of SEQ ID Nos 5, 11, 14, 18, 24, 27, 3, 6, 7, 10, 12, 23 and 25, preferably at least SEQ ID No. 27. The detection of glycation in at least one position selected from the sequences of SEQ ID nos 5, 11, 14, 18, 24, 27, 3, 6, 7, 10, 12, 23 and 25, preferably at least SEQ ID No. 27 may prompt more comprehensive screening to analyse the glycation in at least one sequence selected from the sequences with SEQ ID nos 1 to 30 and 44 to 46, and a verification of the diagnosis, according to the invention, by correlation with the HbA_{1c}.

1. Method for non-invasive diagnosis of diabetes, particularly type II diabetes mellitus, wherein the glycation of human plasma proteins is determined in at least one glycation position selected from
Lys 64 in human serum albumin (P02768, SEQ ID No. 31),
Lys 73 in human serum albumin (P02768, SEQ ID No. 31),
Lys 93 in human serum albumin (P02768, SEQ ID No. 31),
Lys 174 in human serum albumin (P02768, SEQ ID No. 31),
Lys 181 in human serum albumin (P02768, SEQ ID No. 31),
Lys 233 in human serum albumin (P02768, SEQ ID No. 31),
Lys 262 in human serum albumin (P02768, SEQ ID No. 31),
Lys 359 in human serum albumin (P02768, SEQ ID No. 31),
Lys 378 in human serum albumin (P02768, SEQ ID No. 31),
Lys 414 in human serum albumin (P02768, SEQ ID No. 31),
Lys 525 in human serum albumin (P02768, SEQ ID No. 31),
Lys 545 in human serum albumin (P02768, SEQ ID No. 31),
Lys 574 in human serum albumin (P02768, SEQ ID No. 31),
Lys 41 in the human Ig kappa chain C region (P01834, SEQ ID No. 32),
Lys 75 in the human Ig kappa chain C region (P01834, SEQ ID No. 32),
Lys 99 in the human Ig kappa chain C region (P01834, SEQ ID No. 32),
Lys 163 in the human fibrinogen beta chain (P02675, SEQ ID No. 33),
Lys 211 in the human fibrinogen beta chain (P02675, SEQ ID No. 33),
Lys 295 in the human fibrinogen beta chain (P02675, SEQ ID No. 33),
Lys 1003 of human alpha-2-macroglobulin (P01023, SEQ ID No. 34),
Lys 1162 of human alpha-2-macroglobulin (P01023, SEQ ID No. 34),
Lys 683 of human serotransferrin (P02787; SEQ ID No. 35),
Lys 50 in the human Ig lambda chain C region (P01842; SEQ ID No. 36),
Lys 120 of human apolipoprotein A-1 precursor (P02647; SEQ ID No. 37),
Lys 131 of human apolipoprotein A-1 precursor (P02647; SEQ ID No. 37),
Lys 141 of human haptoglobin (P00738; SEQ ID No. 38),
Lys 1325 of human complement C3 precursor (P01024; SEQ ID No. 39),
Lys 71 in the human fibrinogen alpha chain (P02671; SEQ ID No. 40),
Lys 581 in the human fibrinogen alpha chain (P02671; SEQ ID No. 40)
in an isolated blood or plasma sample, and a determination of the HbA_{1c} level is carried out, wherein the glycation in the glycation positions is subsequently correlated with the HbA_{1c} level.

2. Method according to claim 1, comprising the steps of:
Separating the plasma proteins of a blood sample,
Performing enzymatic digestion of the plasma proteins,
Determining glycation in at least glycation position
selected from SEQ ID Nos 1 to 30 and 44 to 46, preferably selected from
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 33),
Lys 50 in the human Ig lambda chain C region (P01842; SEQ ID No. 36),
Lys 141 of human haptoglobin (P00738; SEQ ID No. 38)
Lys 93 in human serum albumin (P02768, SEQ ID No. 31),
Lys 181 in human serum albumin (P02768, SEQ ID No. 31),
Lys 233 in human serum albumin (P02768, SEQ ID No. 31),
Lys 378 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in the human Apolipoprotein A-1 (P02647, SEQ ID No. 37),
or of a peptide selected from SEQ ID Nos 1 to 30 and 44 to 46, preferably from SEQ-ID Nos 5, 11, 14, 18, 24 and 27, particularly preferably in SEQ ID No. 27
and determining the HbA_{1c} level, wherein the glycation in the glycation positions is subsequently correlated with the HbA_{1c} level.

3. Method according to claim 2, characterised in that affinity chromatography is carried out after enzymatic digestion to separate glycated peptides and/or for solid phase extraction.

4. Method according to claim 3, characterised in that boric acid chromatography is carried out as the affinity chromatography.

5. Method according to claim 1, characterised in that glycation is determined in at least one glycation position by mass spectrometry, FRET (Förster Resonance Energy Transfer), ELBIA (Enzyme Linked Boronate Immunoassay) or immunoassay.

6. Method according to claim 1, further comprising the determination of the glycation state of at least one further lysine residue selected from the SEQ ID nos 1 to 30 and 44 to 46, preferably from SEQ ID nos 5, 11, 14, 18, 24, 27, 3, 6, 7, 10, 12, 23 and 25 or 11, 14, 18 and 24.

7. Method according to claim 1, comprising the determination of the glycation state of the peptide SEQ ID no. 27.

8. (canceled)

9. Kit for non-invasive diagnosis von diabetes, particularly type II diabetes mellitus, comprising at least one reagent according to claim 15, said reagent having an affinity for at least one glycation position selected from:
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 33),
Lys 50 in the human Ig lambda chain C region (P01842; SEQ ID No. 36),
Lys 141 of human haptoglobin (P00738; SEQ ID No. 38)
Lys 93 in human serum albumin (P02768, SEQ ID No. 31),
Lys 181 in human serum albumin (P02768, SEQ ID No. 31),
Lys 233 in human serum albumin (P02768, SEQ ID No. 31),
Lys 378 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in the human apolipoprotein A-1 (P02647, SEQ ID No. 37),
preferably in a peptide sequence selected from SEQ ID nos 5, 11, 14, 18, 24, 27, 3, 6, 7, 10, 12, 23 and 25, particularly preferably SEQ ID No. 27.

10. Kit according to claim 9, characterised in that the reagent is an antibody, oligonucleotide aptamer or peptide aptamer.

11. Kit according to claim 9, further comprising at least one immobilised boric acid component to enrich glycated proteins and peptides.

12. Kit according to claim 9, comprising an ELBIA (Enzyme Linked Boronate Immunoassay).

13. Use of a biomarker according to claim 14, said biomarker comprising a glycated lysine selected from:
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 33),
Lys 50 in the human Ig lambda chain C region (P01842; SEQ ID No. 36),
Lys 141 of human haptoglobin (P00738; SEQ ID No. 38)
Lys 93 in human serum albumin (P02768, SEQ ID No. 31),
Lys 181 in human serum albumin (P02768, SEQ ID No. 31),
Lys 233 in human serum albumin (P02768, SEQ ID No. 31),
Lys 378 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in the human apolipoprotein A-1 (P02647, SEQ ID No. 37),
or from a sequence selected from SEQ ID nos 5, 11, 14, 18, 24, 27, 3, 6, 7, 10, 12, 23 and 25, preferably SEQ ID no. 27 as biomarkers for the diagnosis of diabetes, particularly type II diabetes mellitus and/or for monitoring a treatment for diabetes, particularly type II diabetes mellitus.

14. Biomarker comprising at least one glycated lysine selected from SEQ ID Nos 1 to 30 and 44 to 46, preferably selected from:
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 33),
Lys 50 in the human Ig lambda chain C region (P01842; SEQ ID No. 36),
Lys 141 of human haptoglobin (P00738; SEQ ID No. 38)
Lys 93 in human serum albumin (P02768, SEQ ID No. 31),
Lys 181 in human serum albumin (P02768, SEQ ID No. 31),
Lys 233 in human serum albumin (P02768, SEQ ID No. 31),
Lys 378 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in the human apolipoprotein A-1 (P02647, SEQ ID No. 37),
and in the glycation positions of sequences according to SEQ ID Nos 5, 11, 14, 18, 24, 27, 3, 6, 7, 10, 12, 23 and 25, preferably SEQ ID No. 27 for the diagnosis von diabetes, particularly type II diabetes mellitus and/or for monitoring a treatment of diabetes, particularly type II diabetes mellitus.

15. Reagent having affinity for at least one antigen, which is formed by a peptide, which comprises at least one of the following lysines selected from SEQ ID nos 1 to 30 and 44 to 46:
Lys 174 in human serum albumin (P02768, SEQ ID No. 31),
Lys 414 in human serum albumin (P02768, SEQ ID No. 31),
Lys 574 in human serum albumin (P02768, SEQ ID No. 31),
Lys 163 in the human fibrinogen beta chain (P02675, SEQ ID No. 33),
Lys 50 in the human Ig lambda chain C region (P01842; SEQ ID No. 36),
Lys 141 of human haptoglobin (P00738; SEQ ID No. 38)
Lys 93 in human serum albumin (P02768, SEQ ID No. 31),
Lys 181 in human serum albumin (P02768, SEQ ID No. 31),
Lys 233 in human serum albumin (P02768, SEQ ID No. 31),
Lys 378 in human serum albumin (P02768, SEQ ID No. 31),
Lys 525 in human serum albumin (P02768, SEQ ID No. 31),
Lys 545 in human serum albumin (P02768, SEQ ID No. 31),
Lys 120 in the human apolipoprotein A-1 (P02647, SEQ ID No. 37),
and/or in the glycation positions of the sequences according to SEQ ID Nos 5, 11, 14, 18, 24, 27, 3, 6, 7, 10, 12, 23 and 25, preferably SEQ ID No. 27, and adjacent sequence sections of the corresponding protein.

16. Reagent according to claim 15, characterised in that the reagent is an antibody, oligonucleotide aptamer or peptide aptamer.


SEQUENCE LISTING (46 sequences)

SEQ ID NO: 1 - protein, 22 aa, Homo sapiens
  MISC_FEATURE (2)..(2): Cysteine may be alkylated
TCVADESAENCDKSLHTLFGDK

SEQ ID NO: 2 - protein, 17 aa, Homo sapiens
  MISC_FEATURE (9)..(9): glycated
SLHTLFGDKLCTVATLR

SEQ ID NO: 3 - protein, 17 aa, Homo sapiens
  MISC_FEATURE (9)..(9): Cysteine may be alkylated
ETYGEMADCCAKQEPER

SEQ ID NO: 4 - protein, 17 aa, Homo sapiens
  MISC_FEATURE (6)..(6): Methionine may be oxidized
ETYGEMADCCAKQEPER

SEQ ID NO: 5 - protein, 19 aa, Homo sapiens
  MISC_FEATURE (6)..(6): Cysteine may be alkylated
AAFTECCQAADKAACLLPK

SEQ ID NO: 6 - protein, 16 aa, Homo sapiens
  MISC_FEATURE (3)..(3): Cysteine may be alkylated
AACLLPKLDELRDEGK

SEQ ID NO: 7 - protein, 15 aa, Homo sapiens
  MISC_FEATURE (7)..(7): glycated
AEFAEVSKLVTDLTK

SEQ ID NO: 8 - protein, 17 aa, Homo sapiens
  MISC_FEATURE (5)..(5): glycated
ADLAKYICENQDSISSK

SEQ ID NO: 9 - protein, 21 aa, Homo sapiens
  MISC_FEATURE (8)..(8): glycated
TYETTLEKCCAAADPHECYAK

SEQ ID NO: 10 - protein, 17 aa, Homo sapiens
  MISC_FEATURE (6)..(6): glycated
VFDEFKPLVEEPQNLIK

SEQ ID NO: 11 - protein, 15 aa, Homo sapiens
  MISC_FEATURE (1)..(1): glycated
KVPQVSTPTLVEVSR

SEQ ID NO: 12 - protein, 10 aa, Homo sapiens
  MISC_FEATURE (1)..(1): glycated
KQTALVELVK

SEQ ID NO: 13 - protein, 16 aa, Homo sapiens
  MISC_FEATURE (4)..(4): glycated
EQLKAVMDDFAAFVEK

SEQ ID NO: 14 - protein, 12 aa, Homo sapiens
  MISC_FEATURE (1)..(1): glycated
KLVAASQAALGL

SEQ ID NO: 15 - protein, 24 aa, Homo sapiens
  MISC_FEATURE (4)..(4): glycated
VQWKVDNALQSGNSQESVTEQDSK

SEQ ID NO: 16 - protein, 19 aa, Homo sapiens
  MISC_FEATURE (14)..(14): glycated
DSTYSLSSTLTLSKADYEK

SEQ ID NO: 17 - protein, 21 aa, Homo sapiens
  MISC_FEATURE (4)..(4): Cysteine may be alkylated
VYACEVTHQGLSSPVTKSFNR

SEQ ID NO: 18 - protein, 18 aa, Homo sapiens
  MISC_FEATURE (3)..(3): glycated
QVKDNENVVNEYSSELEK

SEQ ID NO: 19 - protein, 16 aa, Homo sapiens
  MISC_FEATURE (3)..(3): glycated
IQKLESDVSAQMEYCR

SEQ ID NO: 20 - protein, 19 aa, Homo sapiens
  MISC_FEATURE (1)..(1): glycated
KWDPYKQGFGNVATNTDGK

SEQ ID NO: 21 - protein, 13 aa, Homo sapiens
  MISC_FEATURE (2)..(2): glycated
SKAIGYLNTGYQR

SEQ ID NO: 22 - protein, 16 aa, Homo sapiens
  MISC_FEATURE (15)..(15): glycated
ALLAYAFALAGNQDKR

SEQ ID NO: 23 - protein, 14 aa, Homo sapiens
  MISC_FEATURE (1)..(1): glycated
KCSTSSLLEACTFR

SEQ ID NO: 24 - protein, 17 aa, Homo sapiens
  MISC_FEATURE (7)..(7): glycated
ADSSPVKAGVETTTPSK

SEQ ID NO: 25 - protein, 12 aa, Homo sapiens
  MISC_FEATURE (2)..(2): glycated
AKVQPYLDDFQK

SEQ ID NO: 26 - protein, 10 aa, Homo sapiens
  MISC_FEATURE (1)..(1): glycated
KWQEEMELYR

SEQ ID NO: 27 - protein, 17 aa, Homo sapiens
  MISC_FEATURE (5)..(5): glycated
AVGDKLPECEAVCGKPK

SEQ ID NO: 28 - protein, 17 aa, Homo sapiens
  MISC_FEATURE (5)..(5): glycated
SEETKENEGFTVTAEGK

SEQ ID NO: 29 - protein, 15 aa, Homo sapiens
  MISC_FEATURE (1)..(1): Methionine may be oxidized
MKGLIDEVNQDFTNR

SEQ ID NO: 30 - protein, 16 aa, Homo sapiens
  MISC_FEATURE (6)..(6): glycated
SSSYSKQFTSSTSYNR

SEQ ID NO: 31 - protein, 609 aa, Homo sapiens [sequence omitted]

SEQ ID NO: 32 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 34 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 35 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 36 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 37 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 38 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 39 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 40 - protein, 866 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 41 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 42 - protein, 453 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 43 - protein, 330 aa, Homo sapiens [sequence omitted]

SEQ ID NO: 44 - protein, 15 aa, Homo sapiens
  MISC_FEATURE (3)..(3): methionine may be oxidized
AVMDDFAAFVEKCCK

SEQ ID NO: 45 - protein, 23 aa, Homo sapiens
  MISC_FEATURE (10)..(10): Glycated
LVNEVTEFAKTCVADESAENCDK

SEQ ID NO: 46 - protein, 15 aa, Homo sapiens
  MISC_FEATURE (3)..(3): Methionine may be oxidized
AVMDDFAAFVEKCCK